(19) United States
(12) Patent Application Publication
(10) Pub. No.: US 2025/0152018 A1
(43) Pub. Date: May 15, 2025

(54) LASER SYSTEM FOR AN OPTOACOUSTIC PROBE

(71) Applicant: Seno Medical Instruments, Inc., San Antonio, TX (US)

(72) Inventor: Xavier Rey Saenz, San Antonio, TX (US)

(73) Assignee: Seno Medical Instruments, Inc., San Antonio, TX (US)

(21) Appl. No.: 18/939,149

(22) Filed: Nov. 6, 2024

Related U.S. Application Data
(60) Provisional application No. 63/597,618, filed on Nov. 9, 2023.

(51) Int. Cl. A61B 5/00

(52) U.S. Cl. CPC A61B 5/0095

(57) ABSTRACT

An optoacoustic probe for optoacoustic imaging of a volume is provided. The optoacoustic probe can include a laser system that has a power supply that can be configured to provide a constant voltage, and a first capacitor bank that may be coupled to the power supply to receive the constant voltage and configured to supply power to a corresponding first flash lamp upon reaching a first determined charge state of the first capacitor bank. The first capacitor bank can include a first closed loop feedback configured with a first setpoint that determines a first charge time for the first determined charge state, and the first flash lamp can be configured to emit a first light along a light path. The optoacoustic probe can also include an optical window that may be configured to carry the first light along the light path to the volume.

### CROSS REFERENCE TO RELATED APPLICATIONS

This application claims benefit to U.S. Provisional Application No. 63/597,618, filed on Nov. 9, 2023, titled “LASER SYSTEM FOR AN OPTOACOUSTIC PROBE”, the complete subject matter of which is expressly incorporated herein by reference in its entirety.

### TECHNICAL FIELD

The present invention relates in general to the field of medical imaging, and in particular to a system relating to optoacoustic imaging.

### BACKGROUND

Optoacoustic imaging systems visualize thin tissue slices noninvasively through skin at a tissue site. A tissue site may contain a variety of tissue structures that may include, for example, tumors, blood vessels, tissue layers, and components of blood. In optoacoustic imaging systems, laser generated light is used to deliver optical energy to a planer slice of the tissue site, which as a result of optical absorption with the tissue structures, produces acoustic waves. An image spatially representing the tissue site can be generated by performing image reconstruction on acoustic signals that return to an ultrasound transducer array. Because biological tissue scatters impinging optical energy in many directions the optical energy can be absorbed by tissue structures outside of a targeted region, which can generate acoustic return signals that interferes with the imaging of tissue structures within the targeted region.

In a flash lamp pumped solid state laser, the intensity of the laser output is proportional to the intensity of the light discharged across the flash lamps, which is controlled by a capacitor bank voltage. In certain applications such as photoacoustics, multiple laser outputs are generated successively using different laser systems involving multiple capacitor banks and power supplies. In high repetition-rate applications such as photoacoustics, the charge time becomes a critical parameter in maintaining the desired frame rate. As the charge time of the capacitor is directly related to the output power (j/sec) of the power supply, a high-power output is often needed.

A need therefore exists for a laser system that provide for better control and output when undergoing a high repetition-rate application such as when being utilized in an optoacoustic probe.

### BRIEF SUMMARY

New and useful systems, apparatuses, and methods for providing optoacoustic imaging are set forth in the appended claims. Illustrative embodiments are also provided to enable a person skilled in the art to make use of the claimed subject matter.

Objectives, advantages, and a preferred mode of making and using the claimed subject matter may be understood best by reference to the accompanying drawings in conjunction with the following detailed description of illustrative embodiments.

In accordance with embodiments herein, an optoacoustic probe for optoacoustic imaging of a volume is provided. The optoacoustic probe can include a laser system that has a power supply that can be configured to provide a constant voltage, and a first capacitor bank that may be coupled to the power supply to receive the constant voltage and configured to supply power to a corresponding first flash lamp upon reaching a first determined charge state of the first capacitor bank. The first capacitor bank can include a first closed loop feedback configured with a first setpoint that determines a first charge time for the first determined charge state, and the first flash lamp can be configured to emit a first light along a light path. The optoacoustic probe can also include an optical window that may be configured to carry the first light along the light path to the volume.

Optionally, the constant voltage can be a first over voltage input for the first capacitor bank relative to the first setpoint of the first closed loop feedback. In one aspect, the laser system can also include a second capacitor bank that can be coupled to the power supply to receive the constant voltage and can be configured to supply power to a corresponding second flash lamp upon reaching a second determined charge state of the second capacitor bank. The second capacitor bank may include a second closed loop feedback configured with a second setpoint that determines a second charge time for the second determined charge state, and the second flash lamp may be configured to emit a second light along the light path. In another aspect, the first setpoint can be different than the second setpoint. In one example, the constant voltage can be a second over voltage input for the second capacitor bank relative to the second setpoint of the second closed loop feedback. Optionally, the first light of the first flash lamp can have a first wavelength, and the second light of the second flash lamp can have a second wavelength that is different than the first wavelength. In yet another example, the optical window can be further configured to carry the second light along the light path to the volume. Alternatively, the first setpoint can be independent of the second setpoint. In another embodiment, the optoacoustic probe can also include an ultrasound transducer covered by an acoustic lens for receiving signals related to the first light carried to the volume.

In accordance with embodiments herein, a method for imagining a volume using an optoacoustic probe is provided that can include supplying a constant voltage from a power supply to a first capacitor bank having a first flash lamp that emits a first light along a light pathway upon reaching a first determined charge state, and providing a first closed loop feedback using a first setpoint that is a first voltage that is less than the constant voltage. The first determined charge time can be based on the first setpoint, and the method can also include carrying the first light along the light pathway to an optical window of the optoacoustic probe.

Optionally, the method can include supplying the constant voltage from the power supply to a second capacitor bank having a second flash lamp that emits a second light along the light pathway upon reaching a second determined charge state, and providing a second closed loop feedback using a second setpoint that is a second voltage that is less than the constant voltage. The second determined charge time can be based on the second setpoint, and the method can also include carrying the second light along the light pathway to the optical window of the optoacoustic probe. In one aspect, the first setpoint can be different than the second setpoint. In another aspect, the first light of the first flash lamp can have a first wavelength, and the second light of the second flash lamp can have a second wavelength that is different than the first wavelength. In one example, the first setpoint may be independent of the second setpoint.

In accordance with embodiments herein, a laser system for an optoacoustic probe is provided that includes a power supply that can be configured to provide a constant voltage, a first capacitor bank that can be coupled to the power supply to receive the constant voltage and can be configured to supply power to a corresponding first flash lamp upon reaching a first determined charge state of the first capacitor bank. The first capacitor bank can include a first closed loop feedback configured with a first setpoint that determines a first charge time for the first determined charge state, and the first setpoint can have a first voltage that is less than the constant voltage.

Optionally, the laser system can also include a second capacitor bank coupled to the power supply to receive the constant voltage and configured to supply power to a corresponding second flash lamp upon reaching a second determined charge state of the second capacitor bank, and the second capacitor bank can include a second closed loop feedback configured with a second setpoint that determines a second charge time for the second determined charge state. In addition, the second setpoint may have a second voltage that is less than the constant voltage. In one aspect, the first setpoint may be different than the second setpoint. In another aspect, the first light of the first flash lamp can have a first wavelength, and the second light of the second flash lamp can have a second wavelength that is different than the first wavelength. In one example, the first setpoint may be independent of the second setpoint. In another example, varying the first setpoint can vary the first charge time without varying the constant voltage of the power supply.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1 shows a schematic block diagram illustrating an embodiment of a combined optoacoustic and ultrasound system that may be used as a platform for the methods and devices disclosed herein.

FIG. 2 shows a schematic orthogonal view of an embodiment of a probe that may be used in connection with the methods and other devices disclosed herein.

FIG. 3 shows a schematic block diagram of a laser system for an optoacoustic probe that may be used in connection with the methods and other devices disclosed herein.

FIG. 4 shows a graph of capacitor voltage over time constants as disclosed herein.

FIG. 5 shows a graph of capacitor current over time constants as disclosed herein.

FIG. 6 shows a schematic block flow diagram of a method for powering a laser system that may be used in connection with the systems and devices disclosed herein.

FIG. 7 shows a graph of capacitor voltage over time as disclosed herein.

FIG. 8 shows a graph of capacitor voltage over time as disclosed herein.

FIG. 9 shows a graph of capacitor voltage over time as disclosed herein.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments, but not other embodiments.

The systems and methods are described below with reference to, among other things, block diagrams, operational illustrations and algorithms of methods and devices to provide optoacoustic imaging with out-of-plane artifact suppression. It is understood that each block of the block diagrams, operational illustrations and algorithms and combinations of blocks in the block diagrams, operational illustrations and algorithms, can be implemented by means of analog or digital hardware and computer program instructions.

These computer program instructions can be stored on computer-readable media and provided to a processor of a general-purpose computer, special purpose computer, ASIC, or other programmable data processing apparatus, such that the instructions, which execute via the processor of the computer or other programmable data processing apparatus, implements the functions/acts specified in the block diagrams, operational block or blocks and or algorithms.

In some cases, frequency domain-based algorithms require zero or symmetric padding for performance. This padding is not essential to describe the embodiment of the algorithm, so it is sometimes omitted from the description of the processing steps. In some cases, where padded is disclosed in the steps, the algorithm may still be carried out without the padding. In some cases, padding is essential, however, and cannot be removed without corrupting the data.

In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Reference will now be made in more detail to various embodiments of the present invention, examples of which are illustrated in the accompanying figures. As will be apparent to one of skill in the art, the data structures and processing steps described herein may be implemented in a variety of other ways without departing from the spirit of the disclosure and scope of the invention herein and should not be construed as being limited to the embodiments set forth herein. Rather, these embodiments are provided so that this disclosure will be thorough and complete, and will fully convey the concept of the disclosure to those skilled in the art.

Embodiments herein may be implemented in connection with one or more of the systems and methods described in one or more of the following patents, publications and/or published applications, all of which are expressly incorporated herein by reference in their entireties:
U.S. Pat. No. 7,999,161, titled “Laser-Activated Nanothermolysis Of Cells” filed Jul. 23, 2007;
U.S. Pat. No. 9,289,191, titled “System and method for Acquiring Optoacoustic Data and Producing Parametric Maps Thereof”, and filed Jun. 13, 2012;
U.S. Pat. No. 9,517,055, titled “System And Method For Acquiring Optoacoustic Data And Producing Parametric Maps Using Subband Acoustic Compensation” filed Nov. 25, 2013;
U.S. Pat. No. 9,724,072, titled “System And Method For Mixed Modality Acoustic Sampling” filed Dec. 13, 2013;
U.S. Pat. No. 9,456,805, titled “System And Method For Acquiring Optoacoustic Data And Producing Parametric Maps Using Interframe Persistent Artifact Removal” filed Dec. 19, 2013;
U.S. Publication 2016/0199037, titled “System And Method For Acquiring Optoacoustic Data And Producing Parametric Maps thereof” filed Mar. 22, 2016;
U.S. Publication 2017/0035388, titled “System And Method For Mixed Modality Acoustic Sampling” filed Oct. 18, 2016;
U.S. Pat. No. 9,792,686, titled “System And Method For Acquiring Optoacoustic Data And Producing Parametric Maps Using Subband Acoustic Compensation” filed Nov. 17, 2016;
U.S. Publication 2017/0296151, titled “System And Method For Mixed Modality Acoustic Sampling” filed Jun. 30, 2017;
U.S. Publication 2013/0109950, titled “Handheld Optoacoustic Probe” filed Nov. 2, 2011;
U.S. Publication 2016/0296121, titled “Handheld Optoacoustic Probe” filed May 2, 2016;
U.S. Pat. No. 8,686,335, titled “System And Method For Adjusting The Light Output Of An Optoacoustic Imaging System” filed Dec. 31, 2011;
U.S. Pat. No. 9,528,936, titled “System And Method For Adjusting The Light Output Of An Optoacoustic Imaging System” filed Mar. 31, 2014;
U.S. Publication 2017/0108429, titled “System And Method For Adjusting The Light Output Of An Optoacoustic Imaging System” filed Dec. 27, 2016;
U.S. Pat. No. 9,330,452, titled “Statistical Mapping In An Optoacoustic Imaging System” filed Mar. 11, 2013;
U.S. Pat. No. 9,836,838, titled “Statistical Mapping In An Optoacoustic Imaging System” filed May 3, 2016;
U.S. Publication 2018/0061050, titled “Statistical Mapping In An Optoacoustic Imaging System” filed Nov. 6, 2017;
U.S. Pat. No. 9,610,043, titled “System And Method For Producing Parametric Maps Of Optoacoustic Data” filed Jun. 13, 2012;
U.S. Publication 2017/0100040, titled “System And Method For Producing Parametric Maps Of Optoacoustic Data” filed Dec. 21, 2016;
U.S. Publication 2013/0338501, titled “System And Method For Storing Data Associated With The Operation Of A Dual Modality Optoacoustic/Ultrasound System” filed Jun. 13, 2012;
U.S. Publication 2013/0338475, titled “Optoacoustic Imaging System With Fiber Optic Cable” filed Jun. 13, 2012;
U.S. Publication 2014/0194723, titled “Multi-Layer Coating For Optoacoustic Probe” filed Jan. 13, 2014;
U.S. Publication 2017/0150890, titled “Optoacoustic Probe With Multi-Layer Coating” filed Jan. 31, 2017;
U.S. Pat. No. 9,615,750, titled “Methods And Compositions For Carrier Agents And Clearing Agents Used In Optoacoustic Imaging Systems” filed Jun. 14, 2012;
U.S. Publication 2013/0116538, titled “Optoacoustic Imaging Systems And Methods With Enhanced Safety” filed Oct. 19, 2012;
U.S. Publication 2015/0297090, titled “Optoacoustic Imaging Systems And Methods With Enhanced Safety” filed Jan. 23, 2015;
U.S. Publication 2013/0289381, titled “Dual Modality Imaging System For Coregistered Functional And Anatomical Mapping” filed Nov. 2, 2012;
U.S. Pat. No. 9,757,092, titled “Method For Dual Modality Optoacoustic Imaging” filed Nov. 2, 2012;
U.S. Publication 2014/0039293, titled “Optoacoustic Imaging System Having Handheld Probe Utilizing Optically Reflective Material” filed Jan. 22, 2013;
U.S. Publication 2017/0014101, titled “Dual Modality Imaging System For Coregistered Functional And Anatomical Mapping” filed Sep. 27, 2016;
U.S. Publication 2013/0303875, titled “System And Method For Dynamically Varying The Angle Of Light Transmission In An Optoacoustic Imaging System” filed Nov. 2, 2012;
U.S. Pat. No. 9,445,785, titled “System And Method For Normalizing Range In An Optoacoustic Imaging System” filed Dec. 21, 2012;
U.S. Pat. No. 9,282,899, titled “System And Method For Detecting Anomalous Channel In An Optoacoustic Imaging System” filed Dec. 21, 2012;
U.S. Publication 2014/0005544, titled “System And Method For Providing Selective Channel Sensitivity In An Optoacoustic Imaging System” filed Dec. 21, 2012;
U.S. Publication 2016/0317034, titled “System And Method For Providing Selective Channel Sensitivity In An Optoacoustic Imaging System” filed Jul. 11, 2016;
U.S. Pat. No. 9,445,786, titled “Interframe Energy Normalization In An Optoacoustic Imaging System” filed Jan. 22, 2013;
U.S. Publication 2017/0000354, titled “Interframe Energy Normalization In An Optoacoustic Imaging System” filed Sep. 19, 2016;
U.S. Publication 2014/0206978, titled “Probe With Optoacoustic Isolator” filed Jan. 22, 2013;
U.S. Pat. No. 9,743,839, titled “Playback Mode In An Optoacoustic Imaging System” filed Mar. 15, 2013;
U.S. Publication 2017/0332916, titled “Playback Mode In An Optoacoustic Imaging System” filed Jul. 27, 2017;
U.S. Pat. No. 9,398,893, titled “System And Method For Diagnostic Vector Classification Support” filed Mar. 11, 2014;
U.S. Pat. No. 10,026,170, titled “System And Method For Diagnostic Vector Classification Support” filed Jul. 19, 2016
U.S. application Ser. No. 16/022,138, titled “System And Method For Diagnostic Vector Classification Support” filed Jun. 28, 2018;
U.S. Pat. No. 9,730,587, titled “Diagnostic Simulator” filed Mar. 15, 2013;
U.S. Publication 2017/0332915, titled “Diagnostic Simulator” filed Jul. 27, 2017;
U.S. Pat. No. 8,823,928, titled “Light Output Calibration In An Optoacoustic System” filed Mar. 15, 2013;
U.S. Pat. No. 9,163,980, titled “Light Output Calibration In An Optoacoustic System” filed Jul. 11, 2014;
U.S. Pat. No. 9,814,394, titled “Noise Suppression In An Optoacoustic System” filed Mar. 15, 2013;
U.S. Publication 2018/0078144, titled “Noise Suppression In An Optoacoustic System” filed Nov. 13, 2017;
U.S. Pat. No. 9,733,119, titled “Optoacoustic Component Utilization Tracking” filed Mar. 15, 2013;
U.S. Publication 2017/0322071, titled “Optoacoustic Component Utilization Tracking” filed Jul. 27, 2017;
U.S. Publication 2015/0101411, titled “Systems And Methods For Component Separation In Medical Imaging” filed Oct. 13, 2014;
U.S. Publication 2015/0305628, titled “Probe Adapted To Control Blood Flow Through Vessels During Imaging And Method Of Use Of Same” filed Feb. 27, 2015
U.S. Publication 2016/0187481, titled “Opto-Acoustic Imaging System With Detection Of Relative Orientation Of Light Source And Acoustic Receiver Using Acoustic Waves” filed Oct. 30, 2015.

As used herein, the phrase “constant voltage” shall refer to voltage that is provided and either does not vary, or does not vary significantly in response to changes in a circuit. For example, a constant voltage of 1000V may vary minimally between 998V and 1002V based on real world variances in equipment and still be considered a constant voltage. Still, the constant voltage does not vary proportionally or in response to charging or discharging of capacitors in the circuit other than as a result of real world variances. In one example, the variance of the constant voltage is less than 2% from a desired voltage while a voltage or power supply that is providing the voltage is operating.

As used herein, the phrase “determined charge state” shall refer to an amount of charge within a capacitor bank required to discharge to provide current to a corresponding flash lamp. The determined charge state may be expressed in a percentage of charge compared to full capacity of the capacitor bank and may for example be 100%, 99%, 98%, etc. Alternatively, the determined charge state may be expressed in an amount of charge that is reached and not expressed as a percentage. Still, upon reaching the determined charge state, the capacitor bank discharges to provide input to a corresponding flash lamp.

As used herein, the phrase “over voltage” or “over voltage input” shall mean that a setpoint of a closed feedback loop in a capacitor bank has a voltage that is less than a constant supply voltage. So, if a supply voltage is providing a constant voltage of 1000V and the setpoint is provided at 999V or less, the corresponding capacitor bank is being operated with an over voltage.

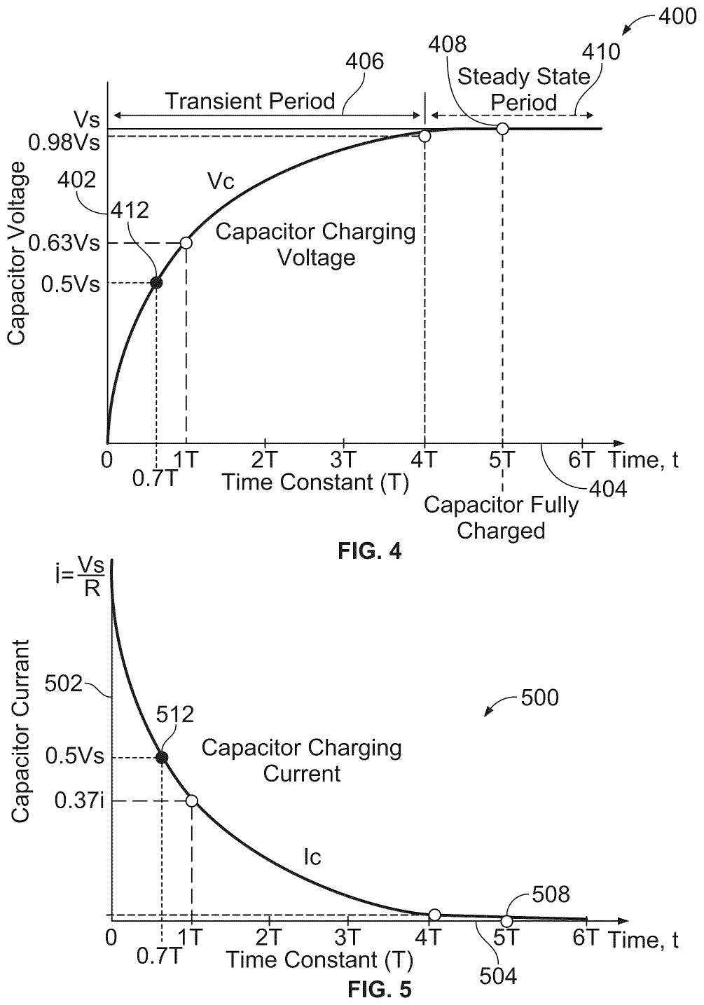

Provided is an optoacoustic system that includes a laser system with different capacitor bank voltage output setpoints using a single power supply. This is accomplished by applying an over voltage input, relative to the desired setpoints, and closed loop feedback. E.g., if capacitor banks receive 1000V (single output from power supply) then the closed loop feedback can be used to set each bank independently of each other, without the need to change the output of the power supply. Because the capacitor charger output current is related to the charge time by Tc=Cload*Vcharge/lout one can see that for high repetition rates a higher output power is often needed. In RC applications one looks at the time constant (T) as seen in FIGS. 4-5, to realize the time needed to reach your voltage setpoint and a steady state. Typically, 5*T is needed for the capacitor to be fully charged. Using the example above, if the capacitor bank receives 1000V, while controlling the desired setpoint of 500V with closed loop feedback, the time needed for the capacitor charge to reach the setpoint becomes 0.7*T, less than ⅕ the original charge time. Thus, speed and efficiencies are greatly enhanced.

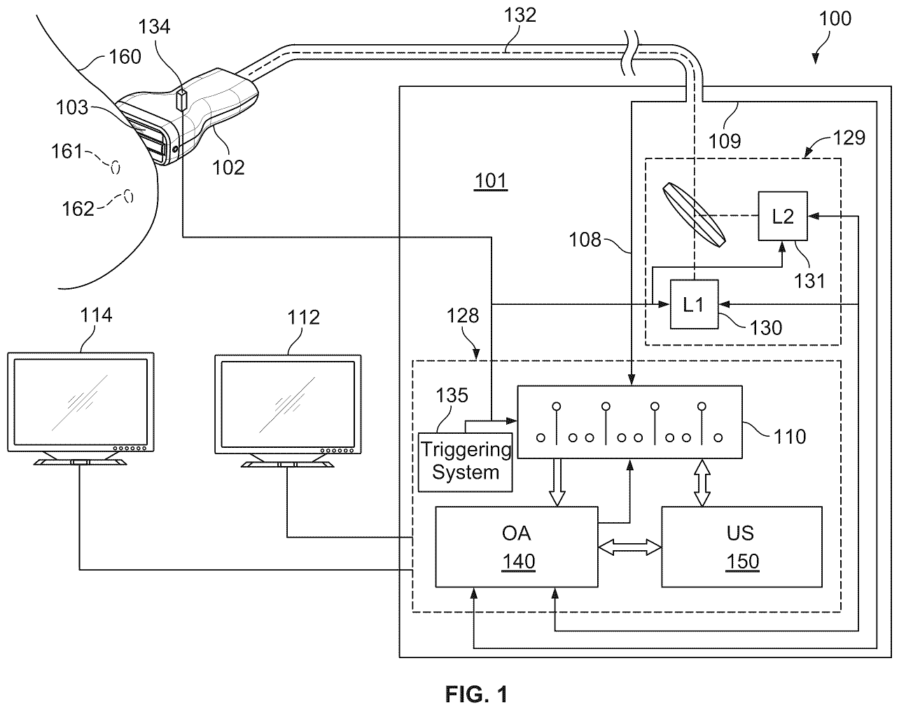

Turning to FIG. 1, generally, device 100 provides an optoacoustic system that may also be employed as multimodality, combined optoacoustic and ultrasound system. In an embodiment, the device 100 includes a probe 102 connected via a light path 132 and an electrical path 108 to a system chassis 101. Within the system chassis 101 is housed a light subsystem 129 and a computing subsystem 128. The computing subsystem 128 includes one or more computing components for ultrasound control and analysis and optoacoustic control and analysis; these components may be separate, or integrated. In an embodiment, the computing subsystem comprises a relay system 110, a triggering system 135, an optoacoustic processing and overlay system 140 and an ultrasound instrument 150. In one embodiment, the triggering system 135 is configured to actuate and control operation of the primary light sources 130, 131 and an auxiliary signal source 134 that in one example is an auxiliary light source. In other examples, the auxiliary signal source may be a sound signal source, piezo based signal source, ultrasound signal source, or the like. The primary light sources 130, 131 are utilized in creating signals for imaging purposes, while the auxiliary signal source 134 is not utilized to create signals for imaging purposes. In an example, the triggering system 135 prevents actuation of the primary light sources 130, 131 before actuation of the auxiliary signal source 134. To this end, the triggering system 135 prevents actuation of the primary light sources 130, 131 until detected light from the auxiliary signal source 134 on a tissue indicates that the probe is contacting the volume 160.

In an embodiment, the light subsystem 129 is capable of producing pulses of light of at least two different wavelengths. In an embodiment, the light subsystem 129 includes two separate primary light sources 130, 131 and an auxiliary signal source 134. In an embodiment the primary light sources 130, 131 are Nd:YAG and Alexandrite lasers. In example embodiments, the auxiliary signal source 134 may be light emitting diode, photodiode, low power laser, or the like. The output of the primary light sources 130, 131 of the light subsystem 129 is delivered to the probe 102 via the light path 132. In another example embodiment, the auxiliary signal source 134 is within the housing of the probe 102 and generates a light that exits the probe 102 through the one or more optical windows 103. Alternatively, the auxiliary signal source 134 is exterior to the probe housing. Specifically, whether within the probe housing or exterior to the probe housing, the auxiliary light source is positioned to emit light on organic tissue, phantom or other volume 160 to cause reflected light that may be received by one or more light detectors. In one example, the light detector is in the distal end of the probe 102 and receives reflected light through the optical window 103.

One or more displays 112, 114, which may be touch screen displays, are provided for displaying images and all or portions of the device 100 user interface. One or more other user input devices (not shown) such as a keyboard, mouse and various other input devices (e.g., dials and switches) may be provided for receiving input from an operator.

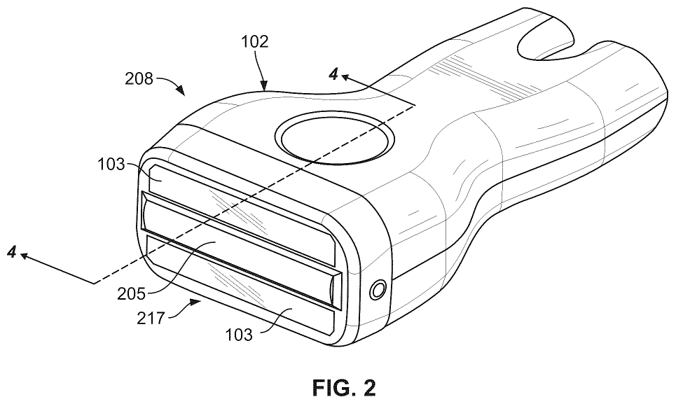

Turning now to FIG. 2, the probe 102 includes an ultrasound transducer covered by an acoustic lens 205. The probe 102 includes distal and proximal ends. A probe face 217 of the probe 102 is at the distal end 208. The probe 102 also includes one or more optical windows 103 through which the light carried on light path 132 can be transmitted to the surface of a volume 160, for example, a three-dimensional volume. Specifically, the probe 102 may be placed in close proximity with organic tissue, phantom or other volume 160 that may have one or more inhomogeneities 161, 162, such as e.g., a tumor, within. An ultrasound gel (not shown) or other material may be used to improve acoustic coupling between the probe 102 and the surface of the volume 160 and/or to improve optical energy transfer. The probe 102, when in proximity with the surface of the volume 160, can emit light from the auxiliary signal source 134 (FIG. 1) onto the surface of the volume 160 that is reflected and detected by one or more light detectors. The computing subsystem 128 may then make determination based on the reflected light regarding whether the probe 102 is contacting the volume 160 using methodologies described herein. Upon the determination of contact between the probe 102 and the volume, the computing subsystem 128 permits actuation of the primary light sources 130, 131 for generating otoacoustical feedback.

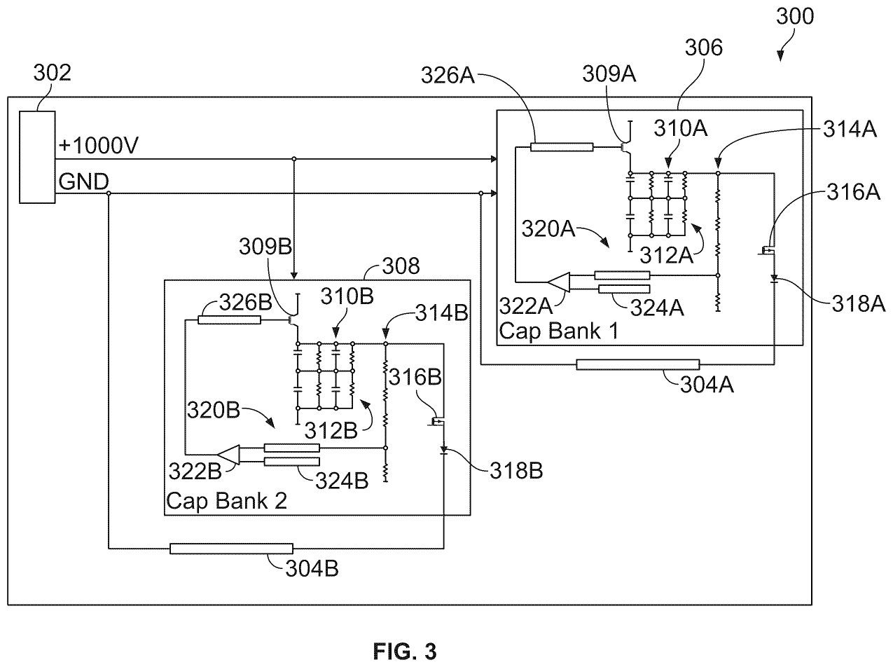

Turning to FIG. 3, a laser system 300 for an optoacoustic probe is illustrated. The laser system 300 can include a power supply 302 such as a battery, generator, etc. that supplies voltage for a flash lamp 304A or 304B via a first capacitor bank 306 (associated with a first flash lamp 304A) and a second capacitor bank 308 (associated with a second flash lamp). In one example, the power supply generates a constant voltage that is an overvoltage of the power needed for powering the flash lamp 304A, 304B. So, in an example, when the voltage required is 750V, the constant overvoltage supplied by the power supply is 1000V. Still, in other examples the power supply may supply a constant voltage of 500V, 750V, 1250V, 2000V, etc. that is dependent on the required voltage needed by the flash lamp 304A, 304B. In addition, while only the first capacitor bank 306 and second capacitor bank 308 are illustrated, in other embodiments additional capacitor banks may be utilized for conditioning the power supplied by the power supply.

The first capacitor bank 306 includes circuitry for conditioning or converting an input from the power supply 302 for use by a corresponding first flash lamp 304A. The circuitry can include a first transistor 309A that receives the input from the power supply 302. The circuitry also includes plural capacitors 310A that are coupled in parallel with plural resistors 312A that function to store and release charge for the circuitry. In parallel to the plural capacitors 310A and plural resistors 312A are auxiliary resistors 314A and a second transistor 316A. The second transistor in one example is an insulated gate bipolar transistor (IGBT), while alternatively the second transistor can be a MOSFET, or other transistor that operates in conjunction with a diode 318A to function as a switch for the circuity that controls when power is supplied to the first flash lamp 304A. In one example, the second transistor 316A and diode 318A operate as a switch and prevent the passage of current to the first flash lamp until a determined charge state of the first capacitor bank is reached. In one example, the determined charge state is 98% charged, while in other examples the determined charge state can be 99% charged, 100% charged, etc. In particular, the determined charge state may be a threshold provided wherein the first capacitor bank 306 is considered fully charged. Meanwhile, the auxiliary resistors 314A provide a pathway to a feedback loop 320A that supplies voltage from the circuitry to a comparator 322A that also receives a setpoint voltage supply 324A such that comparator provides a resulting feedback voltage 326A to the first transistor 309A.

The second capacitor bank 308 can provide the same circuitry as the first capacitor bank 306. In this manner, the second capacitor bank 308 can include circuitry for conditioning or converting an input from the power supply 302 for use by a corresponding second flash lamp 304B. The circuitry can include a first transistor 309B that receives the input from the power supply 302. The circuitry also includes plural capacitors 310B that are coupled in parallel with plural resistors 312B that function to store and release charge for the circuitry. In parallel to the plural capacitors 310B and plural resistors 312B are auxiliary resistors 314B and a second transistor 316B. The second transistor in one example is an insulated gate bipolar transistor (IGBT), while alternatively the second transistor can be a MOSFET, or other transistor that operates in conjunction with a diode 318B to function as a switch for the circuity that controls when power is supplied to the second flash lamp 304B. In one example, the second transistor 316B and diode 318B operate as a switch and prevent the passage of current to the second flash lamp until a determined charge state of the second capacitor bank is reached. In one example, the determined charge state is 98% charged, while in other examples the determined charge state can be 99% charged, 100% charged, etc. In particular, the determined charge state may be a threshold provided wherein the second capacitor bank 308 is considered fully charged. Meanwhile, the auxiliary resistors 314B provide a pathway to a feedback loop 320B that supplies voltage from the circuitry to a comparator 322B that also receives a setpoint voltage supply 324B such that comparator provides a resulting feedback voltage 326B to the first transistor 309B.

As a result, different capacitor bank voltage output setpoints 330A and 330B can be provided using a single power supply 302 by applying an over voltage input, relative to the desired setpoints, and closed loop feedback. In one example the capacitor banks 306 and 308 are provided 1000V (single output from power supply 302) and the closed loop feedback can be used to set each capacitor bank 306, 308 independently of each other, without the need to change the output of the power supply. Thus, in one example the first closed loop feedback could be 500V for the first capacitor bank 306, while the second closed loop feedback could be 450V for the second capacitor bank 308. As a result, lower power requirement can be achieved for higher repetition applications, such as use of the flash lamps 304A, 304B for optoacoustic monitoring. In addition, faster charge times are also achieved while a cost reduction is realized because only a single capacitor charger is required. The term charge time as used herein refers to the amount of time between the discharge of a capacitor bank (e.g., when the second transistor and the diode allow the passage of current to the corresponding flash lamp as a result of the circuit reaching the determined charge state) and the capacitor bank reaching the determined charge state for the next discharge. In addition, the feedback can provide improved pulse to pulse output for the flash lamps 304A, 304B improving the overall optoacoustic system.

FIG. 4 illustrates a graph 400 of capacitor voltage 402 over time constants 404. The time constant represents a determined period of time such as a second, ten seconds, a minute, etc. such that if 1T represents ten second, 5T represents fifty seconds. As illustrated, current laser systems that utilize a varying voltage supply and do not utilize an over voltage have a transition period 406 that lasts until 4T is reached and does not result in a fully charged capacitor until point 408 (e.g., at 5T) during steady state period 410. In contrast, based on experiments using the laser system as described in relation to FIG. 3, the fully charged capacitor occurred at point 412 (e.g., 0.7T) or less than one fifth the time to reach the fully charged capacitor state. In addition, the capacitor voltage to reach the fully charged capacitor state is greatly reduced compared to the previous laser systems.

Similarly, FIG. 5 illustrates a graph 500 showing capacitor current 502 over the same time constants 504. As illustrated, using current laser systems, current in not approach zero until point 508 (e.g., at 5T) whereas for the laser system of FIG. 3, current approaches zero at point 512 (e.g., at 0.7T). As a result, improved functioning is provided.

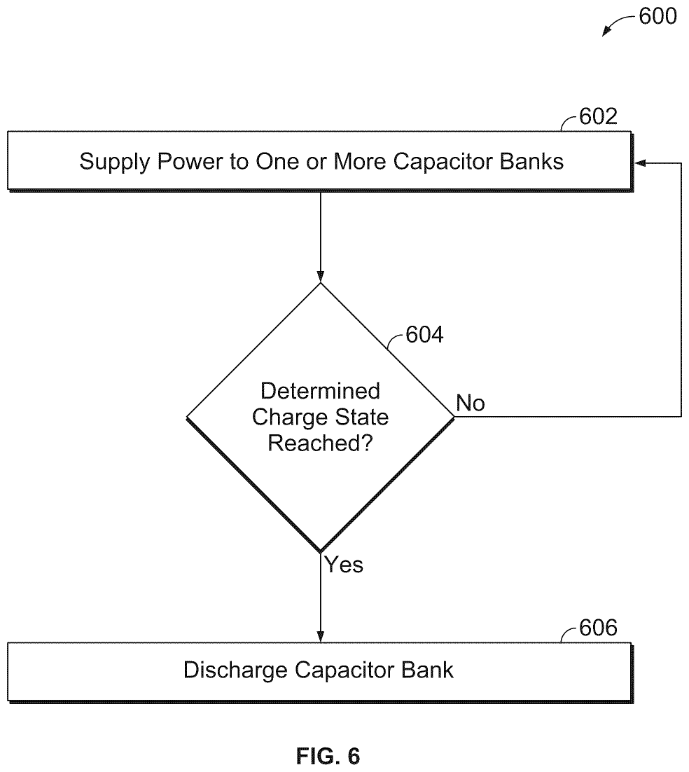

FIG. 6 illustrates a block flow diagram of a method 600 for powering a laser system for an optoacoustic probe. The optoacoustic probe may be an optoacoustic probe illustrated in FIGS. 1-2, and the laser system may be any laser system as illustrated in FIGS. 1 and 3.

At 602, a power supply provides power to one or more capacitor banks. In one example, the power supplied includes a constant voltage that does not vary. In another example, the constant voltage may be an over voltage that is greater than at least one setpoint of a capacitor bank. In one embodiment the constant voltage is 1000V. In another example, two capacitor banks are provided in the laser system. In yet another example, more than two capacitor banks are provided in the laser system.

At 604, a determination is made whether a determined charge state has been reached. In particular, each capacitor bank is utilized to operate a corresponding flash lamp based on feedback from a setpoint. In particular, the constant voltage supplied by the power supply can be greater than a setpoint of each capacitor bank to provide over voltage to each capacitor bank until each capacitor bank reaches a determined charge state (e.g. fully charged). As a result, the period of time for each capacitor bank to become fully charged is reduced compared to currently used laser systems for optoacoustic probes. In one example, when the first capacitor bank reaches a first determined charge state, the first capacitor bank is ready to discharge. Similarly, when a second capacitor bank reaches a second determined charge state, the second capacitor bank is ready to discharge. The first determined charge state and second determined charge state are both representative of a fully charged corresponding capacitor bank. In one example, fully charged does not necessarily mean 100% charged, and instead, a threshold charge percentage, such as 99%, 98%, 95%, etc. can be selected, and once reached, discharge can occur. In this manner, the charge state threshold may be determined prior to use of a capacitor bank, and thus is a determined charge state. In one example, the one or more processors may continuously make determinations related to the state of each of the capacitor banks and may make a determine to vary the determined charge state (e.g., reduce from 99% to 98%) depending on the determinations made. In another example, a switch, such as a transistor is utilized within the circuit, and upon reaching the threshold charge state, the transistor allows the passage of current through the transistor to the flash lamp.

At 606, once a capacitor bank reaches the determined charge state, the capacitor bank discharges to supply power to a corresponding flash lamp. At this point the method continues to repeat during the operation of the laser system until the flash lamp is no longer in use. By providing a laser system that includes a constant supply voltage in combination with setpoints within capacitor banks to provide feedback where the constant voltage is an over voltage, the time for the capacitors of the capacitor banks to reach full charge is greatly reduced while also reducing the voltage to reach full charge. Thus, an improved method is provided.

FIGS. 7-9 illustrate numerous graphs 700, 800, 900 that show capacitor bank voltage 702, 802, 902, over time 704, 804, 904. The first graph 700 illustrates a laser system that utilizes an over voltage input accordingly, to the method of FIG. 6 and the laser system of FIG. 3. In this example, the over voltage input is 600V where a first capacitor bank 706 has a set point voltage of 360V and a second capacitor bank 708 has a set point voltage of 310V. In this example embodiment, the first capacitor bank 706 has an initial charge at 0.5 seconds, and then fires every 200 ms starting at 3.5 seconds. Meanwhile, the second capacitor bank 708 also shows an initial charge at 0.5 second and fires every 200 ms starting at 3.5 seconds. The first graph 700 also illustrates the current draw 710 of the laser system, having a peak current 712 of only 4 amps at 0.5 seconds and only 2 amps during the 200 ms recharge.

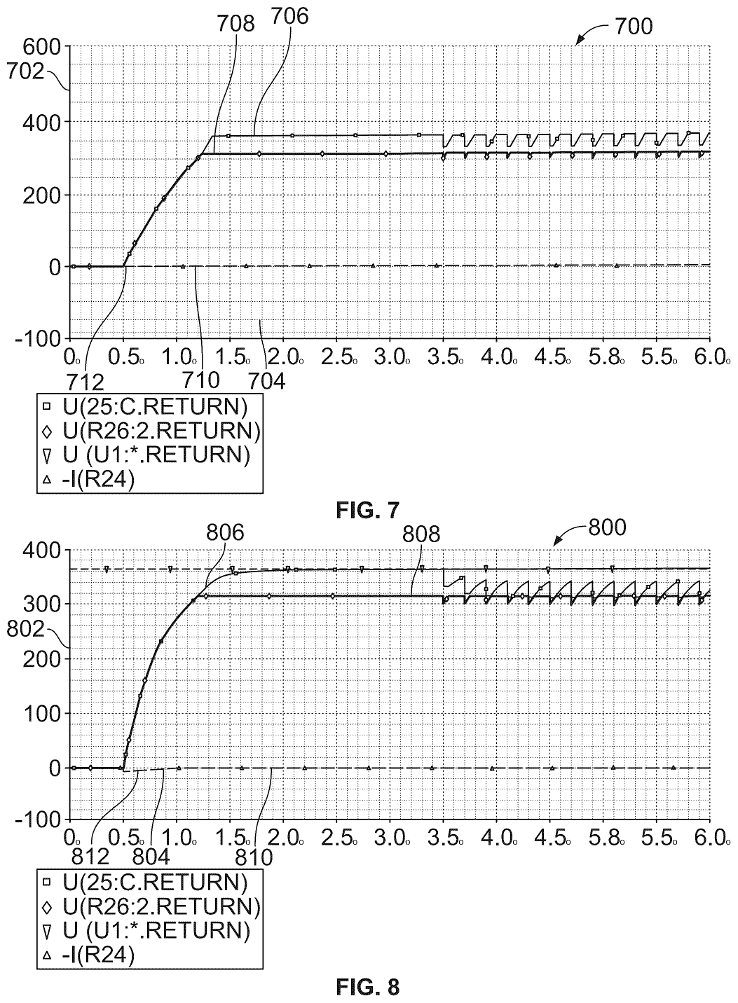

Compare this to the laser system illustrated in FIG. 8 where no overcharge is provided. Again, the first capacitor bank 806 has a setpoint voltage of 360V while the second capacitor bank 808 has a setpoint voltage of 310V. Again, as before, the first capacitor bank 806 and second capacitor bank 808 both show an initial charge at 0.5 seconds and fires every 200 ms starting at 3.5 seconds. In such a system when not providing the overvoltage, the peak current 812 increases to 7 amps as the current draw 810 provides. Thus, as illustrated, the laser system of FIGS. 3 and 6 nearly reduces the peak amps by half. In addition, the first capacitor bank 806 takes an additional 500 ms to reach the initial setpoint compared the method and system described herein. So, when a current limiter is fixed to allow the same initial charge time for the second capacitor bank 808, there is not enough to recharge the first capacitor bank 806 (e.g., the voltage does not reach the desired setpoint once the 200 ms fire sequence is initiated). Thus, the current system and method provide significantly improved results.

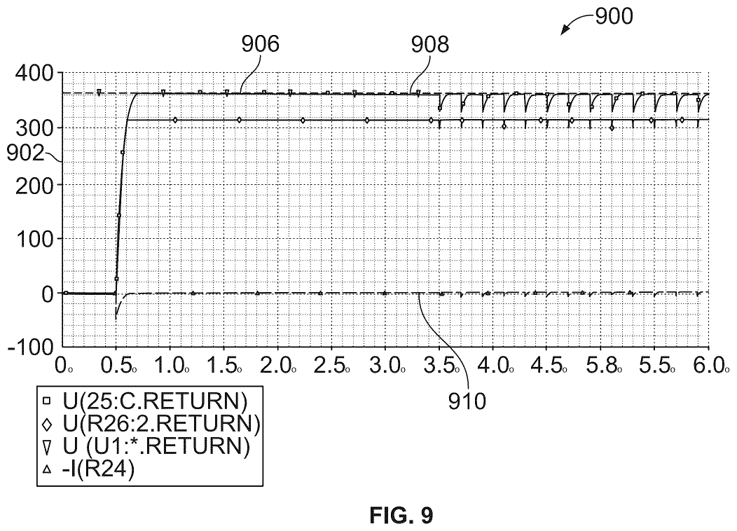

FIG. 9 meanwhile illustrates the identical circuits as FIGS. 7 and 8, only the peak current is set to allow for recharge while firing. So, the first capacitor bank 906 has a setpoint of 360V, the second capacitor bank 908 has a setpoint of 310V, and both the first and second capacitor banks have an initial charge at 0.5 seconds and fire every 200 ms starting at 3.5 seconds. Under this condition, the peak current 912 of the current draw 910, is nearly 16 amps, or almost four times the peak current using the method of FIG. 6 and the laser system of FIG. 3. To this end, without a variable current, the initial current charge could provide an almost 50 amp peak compared to the 4 amps using the method and laser system described herein.

Overall, by utilizing the over voltage methodology described herein, a decreased amount of current consumption is provided compared to a method where a setpoint voltage is provided to maintain the same repetition rate. In addition, the current method and laser system can be provided with reduced cost and complexity compared to set point based systems. To this end, even using a current limiter does not result in the current reduction by providing the overvoltage, despite the cost associated with the current limiter. Thus, an improved system and method are provided.

The present system and methods are described above with reference to block diagrams and operational illustrations of methods and devices comprising an optoacoustic probe. It is understood that each block of the block diagrams or operational illustrations, and combinations of blocks in the block diagrams or operational illustrations, may be implemented by means of analog or digital hardware and computer program instructions. These computer program instructions may be provided to a processor of a general-purpose computer, special purpose computer, ASIC, FPGA or other programmable data processing apparatus, such that the instructions, which execute via the processor of the computer or other programmable data processing apparatus, implements the functions/acts specified in the block diagrams or operational block or blocks. In some alternate implementations, the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved.

As used in this description and in the following claims, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds.

As used in this specification and the appended claims, the term “or” is generally employed in its sense including “and/or” unless the content clearly dictates otherwise.

The recitation herein of numerical ranges by endpoints includes all numbers subsumed within that range (e.g. 1 to 5 includes 1, 1.5, 2, 2.75, 3, 3.80, 4, and 5).

Unless otherwise indicated, all numbers expressing quantities of ingredients, measurement of properties and so forth used in the specification and claims are to be understood as being modified in all instances by the term “about,” unless the context clearly dictates otherwise. Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the scope of the claims, each numerical parameter should at least be construed in light of the number of reported significant digits and by applying ordinary rounding techniques. Any numerical value, however, inherently contains certain errors necessarily resulting from the standard deviations found in their respective testing measurements.

Those skilled in the art will recognize that the methods and systems of the present disclosure may be implemented in many manners and as such are not to be limited by the foregoing example embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

Various modifications and alterations to the invention will become apparent to those skilled in the art without departing from the scope and spirit of this invention. It should be understood that the invention is not intended to be unduly limited by the specific embodiments and examples set forth herein, and that such embodiments and examples are presented merely to illustrate the invention, with the scope of the invention intended to be limited only by the claims attached hereto. Thus, while the invention has been particularly shown and described with reference to a preferred embodiment thereof, it will be understood by those skilled in the art that various changes in form and details may be made therein without departing from the spirit and scope of the invention.

What is claimed is:

1. An optoacoustic probe for optoacoustic imaging of a volume, the optoacoustic probe comprising:
a laser system comprising:
a power supply configured to provide a constant voltage;
a first capacitor bank coupled to the power supply to receive the constant voltage and configured to supply power to a corresponding first flash lamp upon reaching a first determined charge state of the first capacitor bank;
the first capacitor bank including a first closed loop feedback configured with a first setpoint that determines a first charge time for the first determined charge state; and
the first flash lamp configured to emit a first light along a light path; and
an optical window configured to carry the first light along the light path to the volume.

2. The optoacoustic probe of claim 1, wherein the constant voltage is a first over voltage input for the first capacitor bank relative to the first setpoint of the first closed loop feedback.

3. The optoacoustic probe of claim 1, wherein the laser system further comprises:
a second capacitor bank coupled to the power supply to receive the constant voltage and configured to supply power to a corresponding second flash lamp upon reaching a second determined charge state of the second capacitor bank;
the second capacitor bank including a second closed loop feedback configured with a second setpoint that determines a second charge time for the second determined charge state; and
the second flash lamp configured to emit a second light along the light path.

4. The optoacoustic probe of claim 3, wherein the first setpoint is different than the second setpoint.

5. The optoacoustic probe of claim 3, wherein the constant voltage is a second over voltage input for the second capacitor bank relative to the second setpoint of the second closed loop feedback.

6. The optoacoustic probe of claim 3, wherein the first light of the first flash lamp has a first wavelength, and the second light of the second flash lamp has a second wavelength that is different than the first wavelength.

7. The optoacoustic probe of claim 3, wherein the optical window is further configured to carry the second light along the light path to the volume.

8. The optoacoustic probe of claim 3, wherein the first setpoint is independent of the second setpoint.

9. The optoacoustic probe of claim 1, further comprising:
an ultrasound transducer covered by an acoustic lens for receiving signals related to the first light carried to the volume.

10. A method for imagining a volume using an optoacoustic probe comprising:
supplying a constant voltage from a power supply to a first capacitor bank having a first flash lamp that emits a first light along a light pathway upon reaching a first determined charge state;
providing a first closed loop feedback using a first setpoint that is a first voltage that is less than the constant voltage;
wherein the first determined charge time is based on the first setpoint; and
carrying the first light along the light pathway to an optical window of the optoacoustic probe.

11. The method of claim 10, further comprising:
supplying the constant voltage from the power supply to a second capacitor bank having a second flash lamp that emits a second light along the light pathway upon reaching a second determined charge state;
providing a second closed loop feedback using a second setpoint that is a second voltage that is less than the constant voltage;
wherein the second determined charge time is based on the second setpoint; and
carrying the second light along the light pathway to the optical window of the optoacoustic probe.

12. The method of claim 11, wherein the first setpoint is different than the second setpoint.

13. The method of claim 11, wherein the first light of the first flash lamp has a first wavelength, and the second light of the second flash lamp has a second wavelength that is different than the first wavelength.

14. The method of claim 11, wherein the first setpoint is independent of the second setpoint.

15. A laser system for an optoacoustic probe comprising:
a power supply configured to provide a constant voltage;
a first capacitor bank coupled to the power supply to receive the constant voltage and configured to supply power to a corresponding first flash lamp upon reaching a first determined charge state of the first capacitor bank;
the first capacitor bank including a first closed loop feedback configured with a first setpoint that determines a first charge time for the first determined charge state; and
wherein the first setpoint has a first voltage that is less than the constant voltage.

16. The laser system of claim 15, further comprising:
a second capacitor bank coupled to the power supply to receive the constant voltage and configured to supply power to a corresponding second flash lamp upon reaching a second determined charge state of the second capacitor bank;
the second capacitor bank including a second closed loop feedback configured with a second setpoint that determines a second charge time for the second determined charge state; and
wherein the second setpoint has a second voltage that is less than the constant voltage.

17. The laser system of claim 16, wherein the first setpoint is different than the second setpoint.

18. The laser system of claim 16, wherein the first light of the first flash lamp has a first wavelength, and the second light of the second flash lamp has a second wavelength that is different than the first wavelength.

19. The laser system of claim 16, wherein the first setpoint is independent of the second setpoint.

20. The laser system of claim 15, wherein varying the first setpoint varies the first charge time without varying the constant voltage of the power supply.
